(19) United States
(12) Patent Application Publication
(10) Pub. No.: US 2023/0321400 A1
(43) Pub. Date: Oct. 12, 2023

(54) DEFLECTABLE TIP CATHETERS AND RELATED METHODS OF DRUG DELIVERY

(71) Applicant: JANSSEN BIOTECH, INC., Horsham, PA (US)

(72) Inventors: Rudolph CEDRO, Jr., Stockton, NJ (US); Anthony R. DIUBALDI, Jackson, NJ (US); James A. FLEMING, Bethlehem, PA (US); Daniel OLSEN, Califon, NJ (US)

(21) Appl. No.: 18/044,424

(22) PCT Filed: Sep. 2, 2021
(86) PCT No.: PCT/IB2021/058034

§ 371 Date: Mar. 8, 2023

Related U.S. Application Data
(60) Provisional application No. 63/075,403, filed on Sep. 8, 2020.

(51) Int. Cl. A61M 25/01; A61M 25/00

(52) U.S. Cl. CPC A61M 25/0136; A61M 25/0084; A61M 2025/0089; A61B 2017/003

(57) ABSTRACT

The deflectable tip catheters can be used with a primary delivery system (e.g., a bronchoscope, endoscope, or other surgical access device) to access a certain target tissue, such as a tumor (e.g., a lung tumor). The primary access device provides initial access and the deflectable tip catheter can be passed through the primary access device to reach the target tissue which is not otherwise accessible by the primary access device. The deflectable tip catheters have an internal bore free of obstructions such that a drug delivery device (e.g., a needle) or a diagnostic tool can be passed therethrough to directly access the target tissue.

### FIELD

The present disclosure relates generally to devices for navigating passageways in a body, and in particular, to steerable tip catheters that can be used to navigate the tortuous anatomy of body lumens and to provide drug delivery to multiple areas of a target object.

### BACKGROUND

Steerable or deflectable tip catheters are useful in many applications, being a marked improvement over catheters with fixed tip curves. They are especially useful in the treatment and diagnosis of disease states through transluminal access techniques. Steerable or deflectable tip catheters are particularly useful in the fields of interventional cardiology, neurology, and endovascular diagnosis and treatment of disease where access to the disease or treatment site is accomplished through the arterial or venous vasculature. However, some current steerable catheters are guided using puller wires and/or an internal stylet, which can lead to poor lateral compressive strength and transfer of torque to the tip of the catheter.

Additionally, some current steerable catheters are unable to be deflected once within a target site for drug delivery, such as a tumor. Such steerable catheters can only provide drug delivery to a single part of a target site in which it is inserted, and such catheters must be completely removed and reinserted into another target site to provide drug delivery to that target site. Thus, current steerable catheters cannot be deflected or moved once inserted into a drug delivery site, such as tumor. This can decrease the efficiency of a drug delivery to a target site during a procedure.

Accordingly, there remains a need for improved steering tips for use in deflectable tip catheters for drug delivery to target sites with a human body.

### SUMMARY

In general, methods, systems, and devices for deflectable tip catheters are provided.

In one aspect, a deflectable tip catheter is provided. The deflectable tip catheter includes a catheter body and an inner catheter body. The catheter body can include an outer surface and a smooth inner surface. The inner catheter body is coaxially disposed and slidably engaged within the catheter body. The inner catheter body includes a distal tip, a first strut member having a first proximal end and a first distal end and configured to be axially extended and retracted from the catheter body, and a second strut member having a second proximal end and a second distal end. The first strut member and the second strut member are arranged in spaced apart opposition parallel to each other. The first proximal end of the first strut member is integrally formed to the inner catheter body. The first distal end of the first strut member is integrally formed to the distal tip of the inner catheter body, and the second distal end of the second strut member is integrally formed with the tip of the inner catheter body. The second proximal end of the second strut member is integrally formed to the outer surface of the catheter body. The first strut member and the second strut member are configured to deflect while remaining parallel to each other when the first strut member is axially extended and retracted from the catheter body. The deflectable tip catheter is configured to receive a needle to effect drug delivery to a desired target site.

In another aspect, the first strut member and the second strut member of the inner catheter body can be assembled from separable component parts.

In another aspect, the first strut member and the second strut member can be configured to deflect up to about 180°.

In another aspect, the deflectable tip catheter can be configured to be rotated while the first strut member and the second strut member are bent.

In another aspect, the needle can be configured to pass through the inner catheter body while the first strut member and the second strut member are in the deflected configuration.

In another aspect, the inner catheter body can be a substantially tubular structure.

In another aspect, the first strut member and the second strut member have semicircular cross-sectional areas.

In another aspect, the distal tip can be a hollow tubular structure.

In another aspect, the distal tip can include an aperture configured to allow the needle to pass through the distal tip.

In another aspect, the distal tip can be an atraumatic distal tip.

In another aspect, a support structure can be disposed over the inner catheter body and can include a coil-like structure.

In another aspect, a support structure can be disposed over the inner catheter body and can include an axially deflectable tubular structure.

In another aspect, further including an actuator including a proximal end and a distal end, the distal end of the actuator can be fixedly secured to the inner catheter body.

In another aspect, the actuator can be a linear actuator configured to displace the inner catheter body in an axial direction.

In another aspect, the actuator and inner catheter body can be separable.

In another aspect, the actuator and inner catheter body can be made from a single piece of material.

In another aspect, the actuator and inner catheter body can be fabricated from separate component pieces mechanically fastened together.

In another aspect, further including a handle arranged on the inner catheter body, the handle includes an actuator configured to displace the inner catheter body in an axial direction.

In another aspect, the handle is attached to the proximal end of the actuator.

In one aspect, a method of delivering a drug through a deflectable tip catheter is provided. The method includes inserting a tip assembly of the deflectable tip catheter into a target site, the deflectable tip catheter having a catheter body that includes an outer surface and a smooth inner surface, with the inner catheter body being coaxially disposed and slidably engaged within the catheter body, deflecting the tip assembly to align the tip assembly with a specific region within the target site, directing the needle out of the tip assembly and into the specific region within the target site, and delivering a substance through the needle to the specific region within the target site.

In another aspect, the method can further include removing the needle from the specific region, rotating the deflectable tip catheter to arrange the needle at a second specific region within the target site, inserting the needle into the second specific region within the target site, and delivering the substance to the second specific region through the needle.

In one aspect, a method of operating a deflectable tip catheter to deliver a drug is provided. The method includes inserting a tip assembly of the deflectable tip catheter into a target site, directing a needle through the tip assembly, inserting the needle into the target site, deflecting the tip assembly to align the tip assembly with a specific region within the target site, and delivering a substance to the specific region by passing the substance through the needle inserted into the specific region.

In another aspect, the method can further include rotating the deflectable tip catheter in order to arrange the needle at a second specific region within the target site and delivering the substance to the second specific region through the needle.

### BRIEF DESCRIPTION OF DRAWINGS

The present disclosure is described by way of reference to the accompanying figures which are as follows:

FIG. 1 illustrates a steerable catheter device in use with a handle used to navigate a body lumen according to one embodiment of the present disclosure;

FIG. 2 is a side view illustrating a tip assembly of the steerable catheter of FIG. 1;

FIG. 3 is a top view of the tip assembly of FIG. 1;

FIG. 4 is a side view of an inner catheter assembly of the steerable catheter of FIG. 1;

FIG. 5 is a top view of the inner catheter assembly of FIG. 4;

FIG. 6 is a side view of the partial deflection of the tip assembly of the steerable catheter of FIG. 1;

FIG. 7 is a detail view of a portion of the steerable catheter device of FIG. 1;

FIG. 8 is a detail view of a section of the inner catheter assembly of FIG. 4;

FIG. 9 is a perspective view of the inner catheter assembly of FIG. 4;

FIG. 10 is a detail view of a distal end of the inner catheter assembly of FIG. 9;

FIG. 11 is a detail view of a tab of the inner catheter assembly of FIG. 9;

FIG. 12 is a detail view of the distal end of the inner catheter assembly of FIG. 9;

FIG. 13 is a detail view of the tab of the inner catheter assembly of FIG. 9;

FIG. 14 is a detail view of a portion of the inner catheter assembly of FIG. 3;

FIG. 15 is a cross-sectional view of the inner catheter assembly of FIG. 14;

FIG. 16 is a side view depicting the inner catheter assembly of FIG. 2 in use; and

FIG. 17 is a side view depicting the partial deflection of the tip assembly of the inner catheter assembly of FIG. 16 in use.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. A person skilled in the art will understand that the devices, systems, and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can be determined for any geometric shape. A person skilled in the art will appreciate that a dimension may not be a precise value but nevertheless be considered to be at about that value due to any number of factors such as manufacturing tolerances and sensitivity of measurement equipment. Sizes and shapes of the systems and devices, and the components thereof, can depend at least on the size and shape of components with which the systems and devices will be used.

Various exemplary methods, systems, and devices for steerable catheters having a deflectable tip are provided.

The various figures show embodiments of steerable catheters having a deflectable tip of the present disclosure. The devices and related methods are described herein in connection with navigating through a body lumen, including but not limited to an airway, a duct, the GI tract, and vasculature, to gain access to a desired target site (e.g., organs, tissues, tumors, etc.) that are typically difficult to reach. The steerable catheter described herein can be used alone or, more typically, with a primary access device such as a bronchoscope, endoscope, trocar or the like. The primary access device is used to provide access to a region in proximity to the target site, however it is not possible to access the target site using the primary access device. The steerable tip catheter described herein can be used with a primary access device, such as being inserted through a working channel thereof, to reach the desired target site. Once access to the desired treatment site is obtained, a treatment or diagnostic tool can be advanced through the steerable catheter described herein to perform a treatment or provide a diagnostic procedure at the desired target site, for example, positioning a drug delivery at one or more specific locations at a target site. By way of example, the treatment or diagnostic tool can be a needle advanced through the steerable catheter to deliver a drug directly to the target site (e.g., a tumor). Alternatively, the treatment or diagnostic tool can be a biopsy needle or device, or another type of diagnostic or drug delivery tool.

The steerable catheter disclosed herein is particularly useful to provide direct access to the desired target site by a treatment or diagnostic tool that is passed through the steerable catheter. For example, the steerable catheter described herein has joined catheter sections that enable deflection of the catheter tip. The connection between the joined sections is such that the lumen of the steerable catheter is substantially smooth, enabling a treatment and/or diagnostic tool, such as a needle, to be passed through the lumen such that it does not impinge on any connection structures within the lumen. Once the steerable catheter is within the desired target site, a deflectable needle tip can be used to deliver a drug to multiple regions inside of the target site, such as a tumor. Once the deflectable needle is inserted within the tumor, the needle can be deflected in a single plane in order to perform a sweep of at least about 180° within the tumor. At any point during the sweep of the needle within the tumor, a drug can be delivered within the tumor from the needle. In addition to allowing the needle to sweep in a single plane, the steerable catheter can be rotated within the tumor such that the needle is deflected in order to perform a 360° panning of the target site. The size of the target site/tumor can determine the appropriate length of the needle.

A person skilled in the art will understand that in the case of drug delivery, a variety of drugs and/or other pharmaceutical active agents and other substances (including small and large molecules) can be delivered directly to the desired target site through the steerable catheter device described herein. Examples of such substances that can be delivered include oncolytic viruses, antibodies (such as monoclonal antibodies), hormones, antitoxins, substances for the control of pain, substances for the control of thrombosis, substances for the control of infection, peptides, proteins, human insulin or a human insulin analogue or derivative, polysaccharide, DNA, RNA, enzymes, oligonucleotides, anti-allergics, antihistamines, anti-inflammatories, corticosteroids, disease modifying anti-rheumatic drugs, erythropoietin, and vaccines.

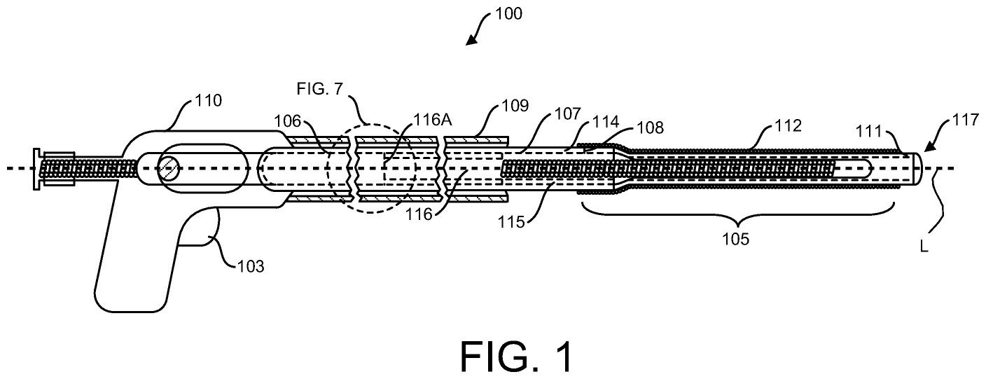

Referring now to the figures, FIG. 1 depicts a catheter assembly 100 used to navigate within a body lumen according to one embodiment of the present disclosure. The catheter assembly can itself be used to navigate directly through a body lumen to access a desired target site or, as discussed above, it can be used with a primary access device. The catheter assembly includes a flexible inner catheter shaft so that the catheter assembly can navigate various body lumens. The catheter assembly includes a deflectable tip, where the deflection is caused by the interaction of an inner catheter tube and an outer catheter tube. In an exemplary implementation, the catheter assembly 100 includes a handle 110 which allows for control of the catheter assembly 100 while being inserted into a body lumen. A catheter sheath 109 extends distally from and is secured to the handle 110 and can be inserted into a body lumen to protect any components within the catheter sheath 109. Once the catheter sheath 109 is positioned just adjacent to or within a body lumen, a catheter shaft 107 is extended from the handle 110 in order to further position the catheter assembly 100 within the body lumen. A tip assembly 105 is arranged coaxially within the catheter shaft 107 and can be moved axially relative to the catheter shaft 107 to deflect the tip assembly 105. An actuator 106 operatively associated with the handle 110 can be used to actuate the tip assembly.

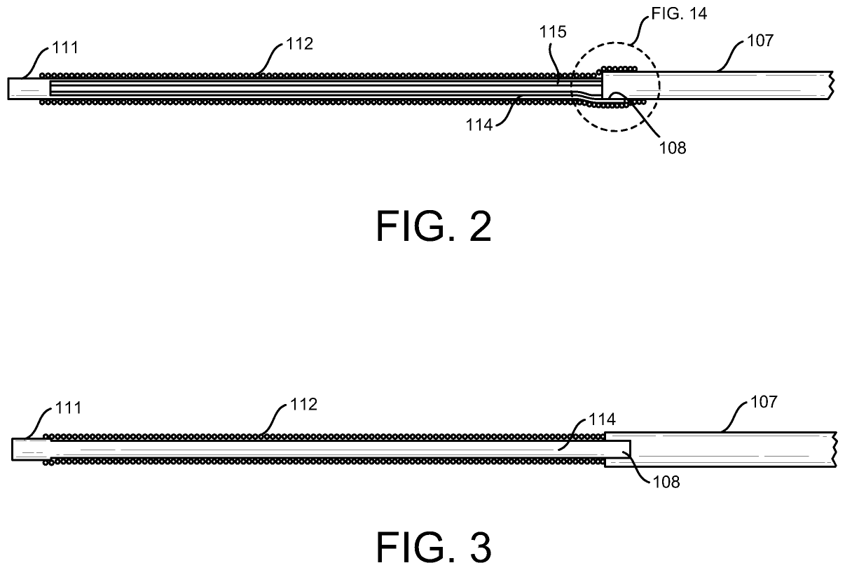

The tip assembly 105 further includes an inner catheter body 111 and support structure 112 coaxially disposed over the inner catheter body 111. The inner catheter body 111 includes a strut 114 and a strut 115, as shown for example in FIG. 2, which are integrally formed with the inner catheter body 111. The support structure 112 acts to constrain the inner catheter body 111 during deflection. The support structure 112 is wrapped around the inner catheter body and has spring-like properties which allow it to deflect from a neutral position when a force is applied, but return to the neutral position when that force is removed. FIG. 1 illustrates the neutral position of the support structure 112 which is aligned with a longitudinal axis L of the catheter assembly 100.

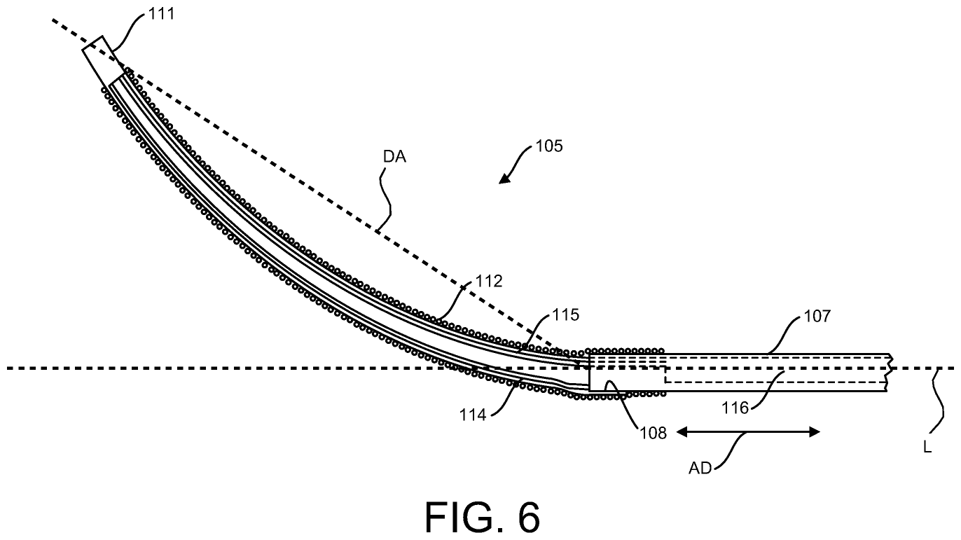

Deflection of the tip assembly can be effected by causing the inner catheter body 111 to deflect. As shown in FIG. 6, the inner catheter body 111 is able to be deflected in one direction or another (i.e., left, right, up, down, or any intermediate positions as a result of rotating the catheter) by adjusting the position of first strut 114 relative to second strut 115. This can be accomplished by moving the inner catheter body 111 axially to change the position of the first strut 114 relative the second strut 115. This is possible as the first strut 114 is secured to a fixed point on the outer catheter shaft 107 while the inner catheter body 111 is free to move axially with respect to the outer catheter shaft 107. When the inner catheter body 111 is in a neutral (i.e., straight or non-deflected) position, both the strut 114 and the strut 115 have portions of substantially equal length extending from the catheter shaft 107. As a result of deflecting the tip assembly 105, the proximal inner catheter body 116 is moved axially (i.e., retracted) along longitudinal axis L within the catheter shaft 107. As the proximal inner catheter body 116 is retracted, the strut 115 is also retracted within the catheter shaft 107 since the strut 115 is integrally formed on the proximal inner catheter body 116. Since the strut 114 is fixed to the outside of the catheter shaft 107, strut 114 does not retract within the catheter shaft 107 as the proximal inner catheter body 116 is retracted. As the strut 115 is retracted, a portion of the strut 115 which previously extended from the catheter shaft 107 is retracted within the catheter shaft 107. As shown in FIG. 6, a deflection occurs within the tip assembly 105 since the lengths of strut 114 and the portion of strut 115 extending from the catheter shaft 107 are no longer substantially equal once the strut 115 is retracted. Deflection in the opposite direction can be accomplished by extending the inner catheter body 116, causing strut 115 to lengthen and assume the outer side of the curve rather than the inner side of the curve as shown in FIG. 6..

In an exemplary embodiment, the tip assembly 105 terminates with an atraumatic distal tip 117 to limit damage and trauma as the catheter assembly 100 is used to navigate the anatomy of the body. In an exemplary implementation, the atraumatic distal tip 117 includes a rounded open end member, as illustrated in FIG. 1, integrated into the distal end of the inner catheter body 111. The atraumatic distal tip 117 can be formed from any one of a number of biocompatible materials known to a person skilled in the art. Exemplary materials include stainless steel, platinum-iridium, and polymers such as polytrifluoroethylene and polyether block amide (e.g., PEBAX).

The catheter shaft 107 is a tube-like biocompatible structure substantially coaxial with catheter sheath 109, and sized such that the actuator 106 is slidably engaged with the catheter shaft 107 so that the actuator 106 can axially move the inner catheter body 111 within and relative to the catheter shaft 107 to deflect the tip assembly 105. In an exemplary implementation, the outer diameter of actuator 106 is smaller than the inner bore diameter of catheter shaft 107, allowing the actuator 106 to slide within the catheter shaft 107.

The catheter sheath 109 is the outermost elongate tube-like structure coaxial with longitudinal axis L and sized to house the tip assembly 105, catheter shaft 107, and actuator 106 during delivery. According to embodiments, a primary function of the catheter sheath 109 is to protect the catheter assembly 100, and to prevent damage to the body lumen or a bronchoscope during use. The catheter sheath 109 can be the outermost layer of a catheter assembly 100 that is inserted within a bronchoscope. When a bronchoscope is used to navigate through a body lumen, the bronchoscope can only be fed through body lumens having a certain maximum diameter, which may present size constraints that will limit the ability to deliver a drug to a specific area. In order to gain access to smaller body lumens, the catheter assembly 100 is used in combination with the bronchoscope, or a similar primary access device. The catheter assembly 100 is fed through the bronchoscope, with the catheter sheath 109 protecting the catheter assembly as it is fed through the bronchoscope. The catheter sheath 109 is operatively coupled at its proximal end to the handle 110, where the handle 110 is used to guide the catheter assembly 100 through the bronchoscope, and/or to provide any necessary manipulations. In one implementation, the catheter assembly 100 need not include the catheter sheath 109. Instead, an introducer sheath not secured to the catheter assembly 100 may be employed to assist delivery of the catheter assembly 100 into the body lumen or the bronchoscope.

In an exemplary implementation, the catheter sheath 109 can be made from various polymeric materials, or combination of polymeric materials known to one of skill in the art. In an embodiment, the outer catheter sheath 109 is constructed from polyethylenes, polyamides, polyurethanes, polytetrafluoroethylenes, or a combination of these materials. Still other polymeric materials may also be used for catheter sheath 109, including, polycarbonates and/or polyimides. In addition, embodiments of the sheath could include reinforcement materials, e.g., metallic braid and/or high tensile strength polymeric braid.

The handle 110 is manipulated by a user to advance the catheter assembly 100 and to deflect the tip assembly 105 in the desired direction. Although handle 110 is shown to be a pistol grip-type handle, a person skilled in the art will appreciate that a variety of other handle types can be used as long as they afford a user the ability to selectively control tip advancement and deflection. As such, a suitable handle will include a mechanism for receiving input from the user, such as trigger 103 shown in FIG. 1, for transferring the user input into linear movement for actuation of tip assembly 105. In an exemplary implementation, the proximal end of the actuator 106 is attached to the handle 110 and provides the mechanism for transferring the linear movement supplied by the handle 110 to the tip assembly 105. The trigger 103 is operatively coupled to the actuator 106 so that when the trigger 103 is moved by a user input, the actuator 106 can axially displace the inner catheter body 111. This axial displacement of the inner catheter body 111 by the actuator 106 causes a deflection of the inner catheter body 111 due to the changing of the position of the strut 114 relative to the position of the strut 115. In another exemplary embodiment, the trigger 103 can be directly coupled to the inner catheter body 111 so that when the trigger 103 is pulled or pushed, the inner catheter body 111 can be axially displaced to cause a deflection of the tip assembly.

The inner catheter body 111 can be a substantially rigid structure made from any one of a number of biocompatible materials known to a person skilled in the art. znon-limiting examples of such materials include surgical stainless steel, Nitinol, or cobalt-chromium alloys. The support structure may come in several forms, but it is typically a coil-like structure capable of readily bending when deflected axially, yet sufficiently rigid to resist radial expansion.

The inner catheter body 111 should have dimensions, including length and diameter, that render it suitable for an intended application. By way of example, the inner catheter body 111 can have a diameter within the range of about 0.005-0.020 millimeters, and preferably within a range of about 0.012-0.015 millimeters. The length of the inner catheter body 111 can vary depending upon the desired application, but it is typically in the range of about 150-180 cm. Additionally, the catheter shaft 107 should have dimensions, including length and diameter, that render it suitable for an intended application. The length of the catheter shaft 107 can vary depending upon the desired application, but it is typically in the range of about 100-120 cm. By way of example, the catheter shaft 107 can have a diameter within the range of about 0.5-5.0 millimeters, and preferably within a range of about 1.0-2.5 millimeters.

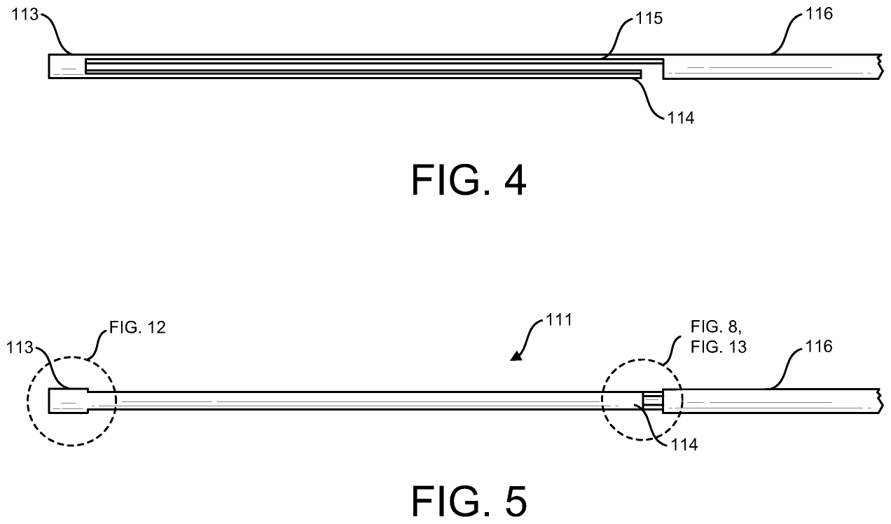

Referring now to FIGS. 2-5, to assist the tip assembly 105 in deflecting, the inner catheter body 111 is comprised of a blunt tip 113 connected at its proximal end to longitudinally arranged strut members, strut 114 and strut 115. In an exemplary implementation, strut 114 and strut 115 are in spaced apart opposition parallel or substantially parallel to one another along longitudinal axis L. The proximal end of the strut 114 terminates at a tab 108. The strut 115 is coupled at its proximal end to the proximal inner catheter body 116. The proximal inner catheter body 116, in turn, is secured to the actuator 106 via connection 116A, which allows the proximal inner catheter body 116 to be moved axially, causing the tip assembly 105 to bend.

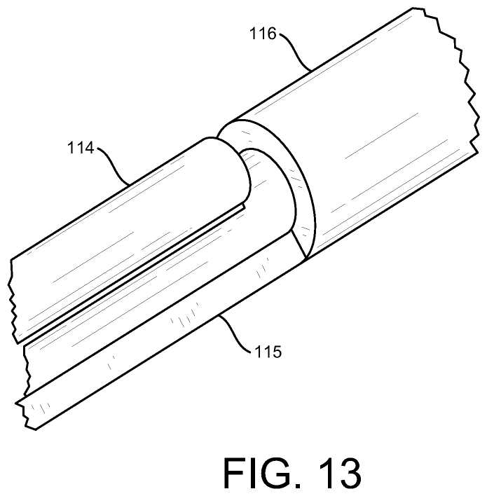

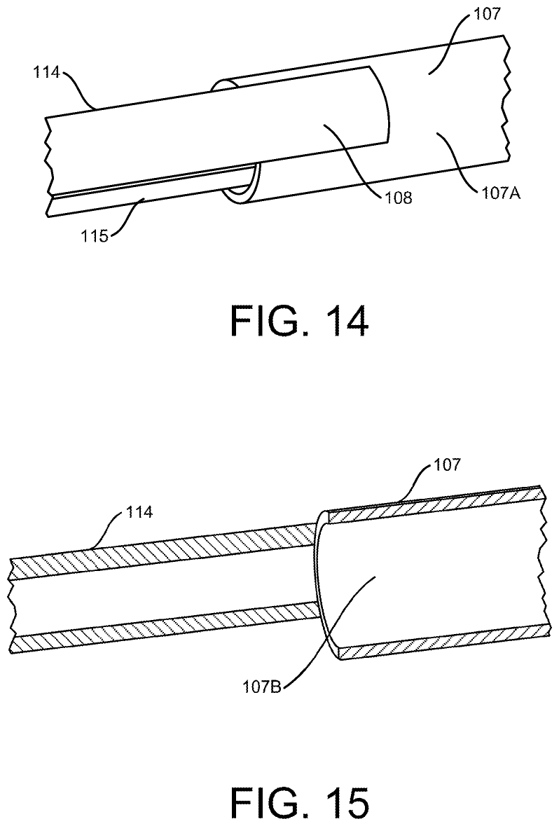

The tab 108, associated with the strut 114, is connected to the catheter shaft 107 as illustrated in FIG. 3 The design through which tab 108 is secured to the outer surface of the catheter shaft 107 is advantageous as it provides a smooth inner surface within the catheter shaft 107, as shown in greater detail in FIGS. 13 and 14. In an exemplary embodiment, due to the tab 108 securing to the outer surface 107A of the catheter shaft 107, a complimentary receptacle formed or cut in the distal end of the catheter shaft 107 is not required. This allows for the inner surface 107B of the catheter shaft 107 to remain smooth since there is no connection formed on the inner surface 107B. Due to the smooth inner surface 107B, any tools (e.g., needles, cameras, tubes) traveling down the working channel of the catheter shaft 107 and inner catheter body 111 will not snag on the securement means of the tab 108 to the catheter shaft 107. One advantage of the smooth inner surface of the catheter shaft 107 is that it helps prevent snags or obstacles formed from the connection between the inner catheter body 111 and the catheter shaft 107. As such, needles and other drug delivery and/or diagnostic devices can be passed through the catheter assembly in a reliable and efficient manner without concern for snagging on any internal structures or protrusions. The tab 108 can be secured to the outer surface of the catheter shaft 107 in various ways including, but not limited to, adhesive, mechanical means (i.e., pinning, crimping), chemical bonding, and heat fusion (e.g., welding, brazing, and soldering).

Referring now to FIG. 6, a partial deflection of the tip assembly 105 is depicted. The catheter shaft 107 is made from a flexible material such that it can navigate through tortuous body lumens while being advanced to a target site. However, the catheter shaft 107 must also have the necessary longitudinal compressive strength to be able to receive translational movement of the proximal inner catheter body 116 via the actuator 106 without undue deformation. In an exemplary implementation, the catheter shaft 107 is made from materials such as stainless steel, a nickel titanium alloy (e.g., Nitinol), or cobalt-chromium alloy, but any material exhibiting the desired characteristics of flexibility and compressive strength may be used.

To deflect the tip assembly 105, the proximal inner catheter body 116 is moved in the axial direction AD within the catheter shaft 107. Since the tab 108 is fixedly secured to the catheter shaft 107, this axial movement of the proximal inner catheter body 116 causes a difference in relative positioning to occur between the strut 114 and the strut 115, with the strut 115 either being extended or retracted relative to the fixed strut 114 from the catheter shaft 107. The strut 115 is not attached to the catheter shaft 107, and is free to move within the catheter shaft 107. The strut 115 of inner catheter body 111 is rigidly attached to the distal end of the proximal inner catheter body 116 such that any movement of the proximal inner catheter body 116 is communicated directly to the strut 115. To deflect the inner catheter body 111, the actuator 106 is translated along the axial direction AD relative to the catheter shaft 107. This movement is communicated to the strut 115, which is free to move relative to the catheter shaft 107. Since the strut 114 of inner catheter body 111 is fixedly attached to the catheter shaft 107, the distal end of the catheter shaft 107 will act as the proximal end point from which the arcuate form of the inner catheter body 111 deflects. This transformation from linear motion to a curved or rotational form is directly proportional to the position of the strut 115 relative to the strut 114 that is exposed from the distal end of the catheter shaft 107. Further extending the exposed strut 115 relative to the strut 114 will deflect the distal end of the catheter assembly 100 in a first direction. Similarly, retracting the exposed length of the strut 115 relative to the strut 114 will deflect the distal end of the catheter assembly 100 in the opposite direction. When the strut 114 and the strut 115 are in a relative neutral position, the distal end of the catheter assembly 100 is substantially straight. Due to the change in relative positioning of the strut 115, a curve forms in the tip assembly 105. The tip assembly 105 curves to a deflection angle DA from the longitudinal axis L when the strut 114 and strut 115 are at different relative positions. The tip assembly 105 can curve to a deflection angle within a range of about 0°-180° for various applications of the tip assembly 105.

For example, as depicted in FIG. 6, the proximal inner catheter body 116 has been retracted along the axial direction AD by the actuator 106 in order to shorten the strut 115 with respect to the strut 114, causing the tip assembly 105 to curve upwards. In order to straighten the tip assembly 105, the actuator 106 only needs to reverse direction along the axial direction AD in order to change the relative position of the strut 115 so that the strut 115 is at an substantially neutral position versus the strut 114. Additionally, this process can be reversed, where the strut 115 is extended by the actuator 106 in order to make the strut 115 extended further than the strut 114. This would cause the tip assembly 105 to curve downward. In an exemplary implementation, the inner catheter body 111 is fabricated to resume a pre-determined configuration when the force providing the translation of the actuator 106 is removed. One material exhibiting shape memory or super-elastic characteristics is Nitinol. Other materials that have shape memory characteristics may also be used, for example, some polymers and metallic composition materials. It should be understood that these materials are not meant to limit the scope of the disclosure. Other biocompatible materials capable of exhibiting similar properties may be suitable.

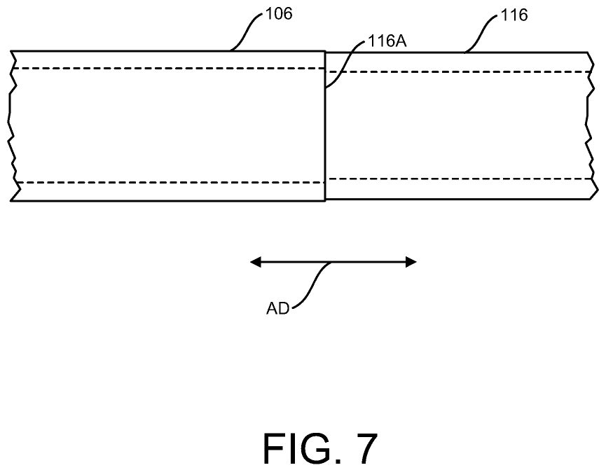

Referring to FIG. 7, the proximal inner catheter body 116 is attached to the actuator 106 at a connection 116A, which can be any type of connection that fixedly secures the actuator 106 to the inner catheter body 116, including, but not limited to, adhesive, mechanical means (i.e., pinning, crimping), or chemical bonding, and heat fusion (e.g., welding, brazing, and soldering). As a result of this connection, the proximal inner catheter body 116 and actuator 106 cannot move relative to one another. This design allows the inner catheter body 111 and the actuator 106 to be made from two separate components. However, the inner catheter body 111 and the actuator may be made as monolithic units from a continuous tube, in which case there would be no attachment point between actuator 106 and proximal inner catheter body 116 at connection 116A. Additionally, the attachment of the proximal end of the actuator 106 to the handle 110 provides the mechanism for transferring the linear movement supplied by the handle 110 to the tip assembly 105. This linear movement in the axial direction AD results in the inner catheter body 111 deflecting the tip assembly 105 up or down, depending on the movement imparted by the handle 110. Accordingly, the actuator 106 must be sufficiently rigid to transmit the linear movement, yet flexible enough to navigate tortuous body lumens during delivery of the steerable catheter through the body. In an exemplary embodiment, the actuator 106 can be made integral with the tip assembly 105. The actuator 106 can be any type of liner actuator including, but not limited to, hydraulic, electromagnetic, or manually actuated. Additionally, the actuator 106 can be controlled via a control unit having a processor, a memory, and a computer implemented function stored on the memory, which can be executed by the processor.

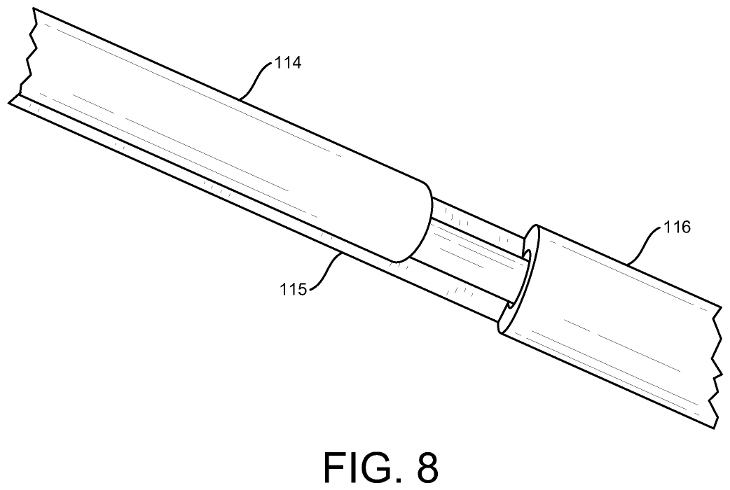

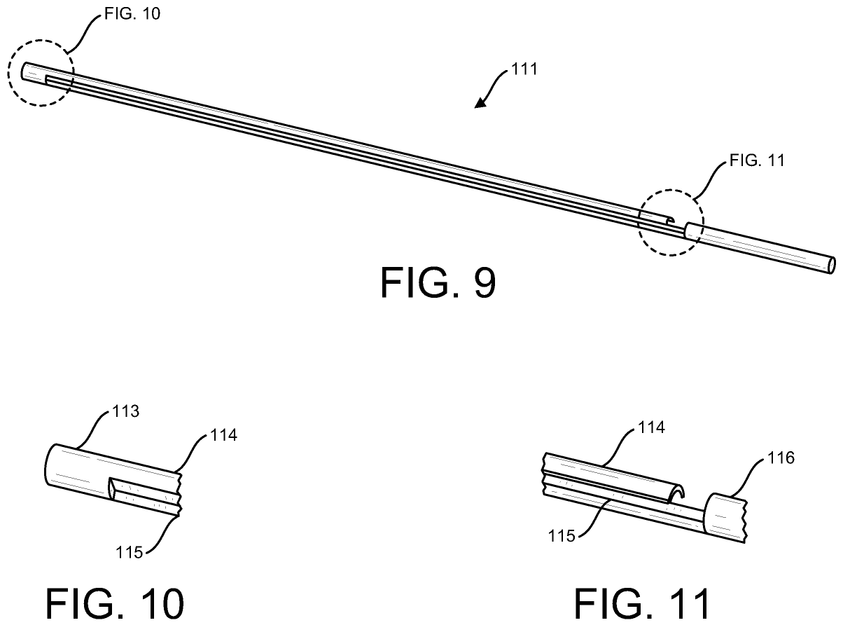

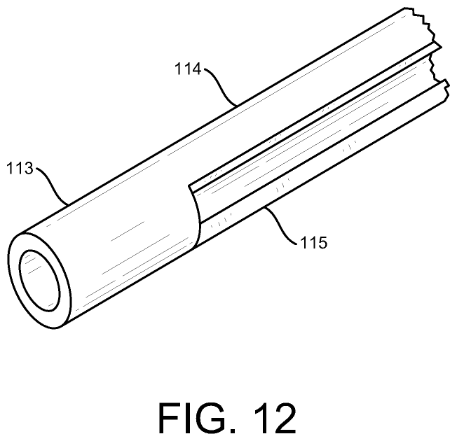

Referring now to FIGS. 8-13, the inner catheter body 111 is shown and described as having separate components (proximal inner catheter body 116, strut 115, strut 114 and tip 113). However, it should be understood that the inner catheter body 111 is broken down into separate components for ease of illustration and explanation. In an exemplary implementation, the proximal inner catheter body 116, strut 115, strut 114 and tip 113 are formed as a monolithic unit from a single piece of material.

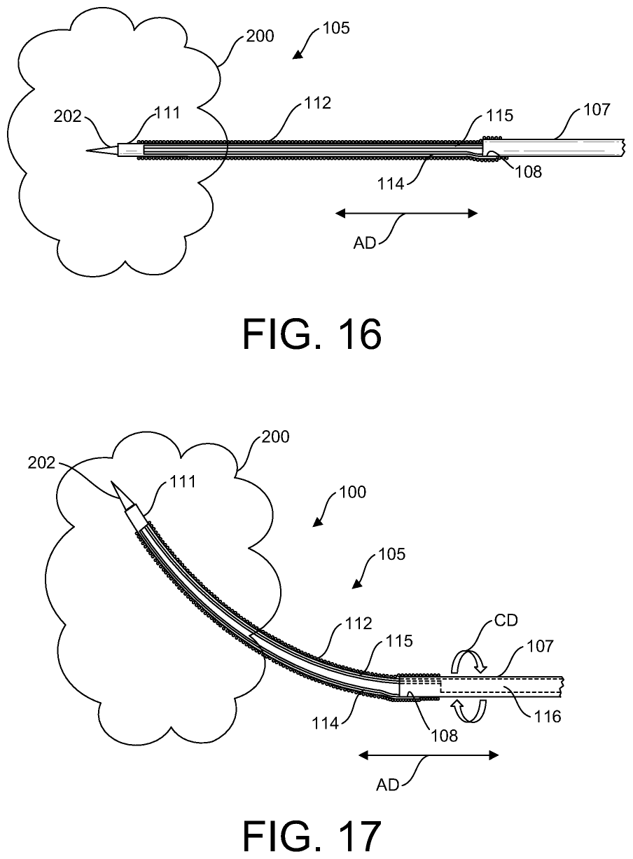

Referring now to FIGS. 16 and 17, the catheter assembly 100 is shown inserted within a desired target site, such as mass 200, during a bronchoscopy procedure such as to access a lung tumor for direct delivery of a drug thereto. A bronchoscopy is a procedure through which a physician can access and examine the airways of a patient. The physician will thread a bronchoscope through a patient's nose or mouth to reach the lungs. Using the bronchoscope, a doctor can view all of the structures that make up a patient's respiratory system, including the larynx, trachea, and the smaller airways of the lungs, which include the bronchi and bronchioles. Once a local anesthetic is applied to the throat of a patient, the doctor will insert the bronchoscope into a patient's nose, for example. The bronchoscope passes from the patient's nose down to the throat until it reaches the bronchi.

As discussed above, the catheter assembly 100 can be used in combination with a primary access device as described above, an example of which is a bronchoscope. In this example the catheter assembly 100 is inserted into a bronchoscope once the bronchoscope has already been inserted within a patient to the bronchus of a patient's lung. Once the bronchoscope is properly positioned, tip assembly 105 of the catheter assembly 100 can be passed through the bronchoscope into the smaller bronchioles of a patient's lung, which branch off from the bronchus. The exemplary implementation shown in FIGS. 16 and 17, in which the bronchoscope is not shown for ease of illustration, the catheter assembly 100 is used to deliver a drug to a tumor (i.e., mass 200) situated within a bronchiole of a patient's lung. After the bronchoscope is properly positioned and the catheter assembly is advanced out of the bronchoscope in the manner discussed above, a needle 202 can be advanced through the catheter shaft 107 and inner catheter body 111. A person skilled in the art will appreciate that the needle can be pre-positioned within the catheter assembly 100 or it can be inserted therethrough after proper positioning of the catheter assembly.

The needle 202 can be a flexible needle in order to follow the curvature of the tip assembly 105. Further, the tip assembly 105 and needle 202 should be sufficiently flexible so as to be able to deflect due to the curvature and limited space within the bronchioles of a lung. In addition, the needle 202 should have dimensions, including length and diameter, that render it suitable for an intended application. By way of example, the needle 202 can have a diameter within the range of 0.5-2.0 millimeters, and preferably a diameter of 1.0 millimeter, such as a 30 gauge needle.

Still referring to FIGS. 16 and 17, once the catheter assembly is properly positioned, the needle 202 is inserted into the mass 200 in the axial direction AD. A person skilled in the art will appreciate that during insertion, the tip assembly 105 is sufficiently rigid to resist any buckling that could result from the axial pushing force exerted on the inner catheter body 111. Once the needle 202 is positioned within the mass 200, the tip assembly 105 is deflected in order to curve the needle 202 upward or downward within the mass 200. It is to be understood that while the catheter tip is described as being deflected either “up” or “down,” the catheter assembly 100 can be rotated 360° in the circumferential direction CD. This allows the needle 202 to deliver a substance in a 360° arc in the mass 200.

In another exemplary embodiment, prior to needle 202 being inserted into the mass 200, the tip assembly 105 is deflected in order to curve the needle 202 upward or downward. Once the catheter assembly 100 is properly positioned, the needle 202 is inserted into the mass 200 in the axial direction AD. Once the needle 202 is positioned within the mass 200, a drug may be delivered within the mass 200 though the needle 202. Once the drug is delivered to a specific location within the mass 200, the needle can be retracted from the mass 200. Then, the tip assembly 105 is deflected in order to curve the needle 202 upward or downward to position the needle 202 at a different location of the mass 200. The catheter assembly 100 can also be rotated 360° in the circumferential direction CD. This allows the needle 202 to deliver a substance in a 360° arc to the mass 200. Once the tip assembly 105 is in the correct position, the needle 202 can be reinserted into the mass 200 in order to provide a drug to another location within the mass 200. This process can be repeated multiple times until the mass 200 has been fully inundated with a drug at multiple locations.

The present disclosure has been described above by way of example only within the context of the overall disclosure provided herein. It will be appreciated that modifications within the spirit and scope of the claims may be made without departing from the overall scope of the present disclosure.

What is claimed is:

1. A deflectable tip catheter, comprising:
a catheter body including an outer surface and a smooth inner surface; and
an inner catheter body coaxially disposed and slidably engaged within the catheter body, the inner catheter body including:
a distal tip;
a first strut member including a first proximal end and a first distal end and configured to be axially extended and retracted from the catheter body; and
a second strut member including a second proximal end and a second distal end, wherein the first strut member and the second strut member are arranged in spaced apart opposition parallel to each other, wherein the first proximal end of the first strut member is integrally formed with the inner catheter body, the first distal end of the first strut member is integrally formed with the tip of the inner catheter body, the second distal end of the second strut member is integrally formed with the distal tip of the inner catheter body, and the second proximal end of the second strut member is integrally formed with the outer surface of the catheter body, wherein the first strut member and the second strut member are configured to deflect while remaining parallel to each other when the first strut member is axially extended and retracted from the catheter body;
wherein the deflectable tip catheter is configured to receive a needle to effect drug delivery to a desired target tissue.

2. The deflectable tip catheter of claim 1, wherein the first strut member and the second strut member of the inner catheter body are assembled from separable component parts.

3. The deflectable tip catheter of claim 1, wherein the first strut member and the second strut member are configured to deflect up to about 180°.

4. The deflectable tip catheter of claim 1, wherein the deflectable tip catheter is configured to be rotated while the first strut member and the second strut member are in a deflected configuration.

5. The deflectable tip catheter of claim 1, wherein the needle is configured to pass through the inner catheter body while the first strut member and the second strut member are deflected..

6. The deflectable tip catheter of claim 1, wherein the inner catheter body is a substantially tubular structure.

7. The deflectable tip catheter of claim 1, wherein the first strut member and the second strut member have semicircular cross-sectional areas.

8. The deflectable tip catheter of claim 1, wherein the distal tip is a hollow tubular structure.

9. The deflectable tip catheter of claim 8, wherein the distal tip includes an aperture configured to allow the needle to pass through the distal tip.

10. The deflectable tip catheter of claim 1, wherein the distal tip is an atraumatic distal tip.

11. The deflectable tip catheter of claim 1, wherein a support structure disposed over the inner catheter body comprises a coil-like structure.

12. The deflectable tip catheter of claim 1, wherein a support structure disposed over the inner catheter body comprises an axially deflectable tubular structure.

13. The deflectable tip catheter of claim 1, further comprising an actuator including a proximal end and a distal end, the distal end of the actuator fixedly secured to the inner catheter body.

14. The deflectable tip catheter of claim 13, wherein the actuator is a linear actuator configured to displace the inner catheter body in an axial direction.

15. The deflectable tip catheter of claim 13, wherein the actuator and inner catheter body are separable.

16. The deflectable tip catheter of claim 13, wherein the actuator and inner catheter body are made from a single piece of material.

17. The deflectable tip catheter of claim 13, wherein the actuator and inner catheter body are fabricated from separate component pieces mechanically fastened together.

18. The deflectable tip catheter of claim 1, further comprising a handle arranged on the inner catheter body, the handle having an actuator configured to displace the inner catheter body in an axial direction.

19. The deflectable tip catheter of claim 18, wherein the handle is attached to the proximal end of the actuator.

20. A method of delivering a drug through a deflectable tip catheter, comprising:
inserting a tip assembly of the deflectable tip catheter into a target site, the deflectable tip catheter having a catheter body that includes an outer surface and a smooth inner surface, with the inner catheter body being coaxially disposed and slidably engaged within the catheter body;
deflecting the tip assembly to align the tip assembly with a specific region within the target site; and
directing the needle out of the tip assembly and into the specific region within the target site; and
delivering a substance through the needle to the specific region within the target site.

21. The method of claim 20, further comprising:
removing the needle from the specific region;
rotating the deflectable tip catheter to arrange the needle at a second specific region within the target site;
inserting the needle into the second specific region within the target site; and
delivering the substance to the second specific region through the needle.

22. A method of operating a deflectable tip catheter to deliver a drug, comprising:
inserting a tip assembly of the deflectable tip catheter into a target site;
directing a needle through the tip assembly;
inserting the needle into the target site;
deflecting the tip assembly to align the tip assembly with a specific region within the target site; and
delivering a substance to the specific region by passing the substance through the needle inserted into the specific region.

23. The method of claim 24, further comprising:
rotating the deflectable tip catheter in order to arrange the needle at a second specific region within the target site; and
delivering the substance to the second specific region through the needle.
